(19) United States
(12) Patent Application Publication
(10) Pub. No.: US 2026/0248932 A1
(43) Pub. Date: Aug. 27, 2026

(54) CATIONIC AMPHIPHILIC POLYPEPTIDE FOR DELIVERING RNA AND METHOD FOR PREPARING THE SAME

(71) Applicant: INHA UNIVERSITY RESEARCH AND BUSINESS FOUNDATION, Incheon (KR)

(72) Inventors: Hyun Jin KIM, Incheon (KR); Jun Su AN, Gwangju (KR)

(73) Assignee: INHA UNIVERSITY RESEARCH AND BUSINESS FOUNDATION, Incheon (KR)

(21) Appl. No.: 18/871,152

(22) PCT Filed: Jun. 26, 2024
(86) PCT No.: PCT/KR2024/008856

§ 371 Date: Dec. 2, 2024

(30) Foreign Application Priority Data
Jun. 30, 2023 (KR) 10-2023-0085134
Jun. 25, 2024 (KR) 10-2024-0082643

(51) Int. Cl. A61K 47/64; A61K 48/00; C07K 14/00

(52) U.S. Cl. CPC A61K 47/6455; A61K 48/0041; C07K 14/001

(57) ABSTRACT

The present disclosure relates to a cationic amphiphilic polypeptide for delivering RNA and a method for preparing the same, wherein it was found that a polypeptide polymer, which is obtained by binding a conjugate in which diethylenetriamine (DET) is bonded to aspartic acid; and a conjugate in which octylamine (OCT) is bonded to aspartic acid in a specific order, exhibits superior mRNA transcription efficiency to a polymer bonded in a different order, such that it may be useful as the polypeptide polymer, a method for preparing the same, or a composition for delivering RNA including the same.

### CROSS REFERENCE TO PRIOR APPLICATIONS

This application is a National Stage Application of PCT International Patent Application No. PCT/KR2024/008856 filed on Jun. 26, 2024, under 35 U.S.C. § 371, which claims priority to Korean Patent Application Nos. 10-2024-0082643 filed on Jun. 25, 2024 and 10-2023-0085134 filed on Jun. 30, 2023, respectively, which are all hereby incorporated by reference in their entirety.

### TECHNICAL FIELD

The present disclosure relates to a cationic amphiphilic polypeptide for delivering RNA and a method for preparing the same.

### BACKGROUND ART

In vitro transcribed (hereinafter referred to as IVT) mRNAs have been progressively developed for use in vaccination, cancer immunotherapy, and genome editing. The success of IVT mRNA therapeutics has been interfered with by the fragility and immunogenicity of single-stranded RNA molecules, as well as inefficient cellular uptake of nucleic acids due to a negatively charged macromolecular structure. Synthetic delivery such as lipid nanoparticles and polymeric nanoparticles (polyplexes) have been developed to protect mRNA from nucleases and enhance internalization into target cells. Recently, cationic polymers have been developed to include amino groups and hydrophobic groups in a random or defined arrangement. Amino groups are responsible for electrostatic associations between cationic polymers and anionic payloads; and endosomal escape of nanoparticles after cellular internalization into target cells/tissues. Hydrophobic groups allow hydrophobic interactions during formation of nanoparticles, which increases the colloidal stability of polymeric nanoparticles in aqueous solutions, such as in vitro and in vivo conditions.

The behavior of the polymer in a solution is highly dependent on the sequence of monomers and its length. The well-defined sequence of the polymer determines the higher-order structure that brings a desired function. Thus, sequence-controlled polymers in which monomer units with different chemical properties are arranged in a sequential manner are able to control the structure-property relationships in polymeric materials more effectively. Quantitative structure-property relationships of sequence-controlled polymers have been understood gradually, including the binding phase separation of the reversely charged polyelectrolyte into complex coacervate. For example, the sequence of charged polyelectrolytes affects the intensity of the electrostatic interaction between the two molecules, resulting in phase separation and coacervate formation, while the sequence-controlled polymer exhibits unique thermal and pH responses that are distinct from that of random copolymers in an aqueous solution. Recently, various polymerization methods such as radical polymerization and supramolecular polymerization are being researched and developed to synthesize sequence-controlled polymers.

### DISCLOSURE OF THE INVENTION

### Technical Goals

An object of the present disclosure is to provide a polypeptide polymer represented by the following Chemical Formula 1.

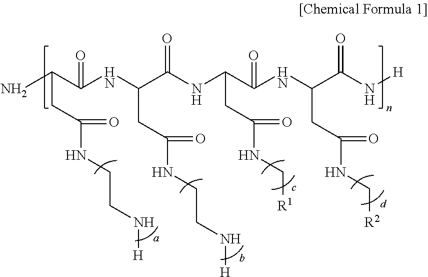

In the Chemical Formula 1, n is one of 1 to 20, a and b may be the same or different [8] and are each independently one of 1 to 5, c and d may be the same or different and are each independently one of 1 to 20, and R¹ and R² may be the same or different and are each independently hydrogen or (C4-C12)cycloalkyl.

Another object of the present disclosure is to provide a composition for delivering RNA, including the polypeptide polymer.

Another object of the present disclosure is to provide a reagent composition for promoting RNA transcription, including the polypeptide polymer.

Another object of the present disclosure is to provide a method for preparing a polypeptide polymer represented by the Chemical Formula 1, the method including: preparing a conjugate in which diethylenetriamine (DET) is bonded to aspartic acid (first step); preparing a conjugate in which octylamine (OCT) is bonded to aspartic acid (second step); and binding the conjugate of the first step and the conjugate of the second step (third step).

### Technical Solutions

To achieve the above object, the present disclosure provides a polypeptide polymer represented by the following Chemical Formula 1.

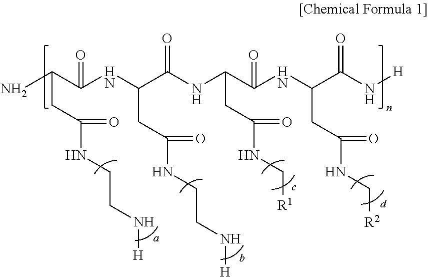

In the Chemical Formula 1, n is one of 1 to 20, a and b may be the same or different and are each independently one of 1 to 5, c and d may be the same or different and are each independently one of 1 to 20, and R¹ and R² may be the same or different and are each independently hydrogen or (C4-C12)cycloalkyl.

In addition, the present disclosure provides a composition for delivering RNA, including the polypeptide polymer.

In addition, the present disclosure provides a reagent composition for promoting RNA transcription, including the polypeptide polymer.

In addition, the present disclosure provides a method for preparing a polypeptide polymer represented by the Chemical Formula 1, the method including: preparing a conjugate in which diethylenetriamine (DET) is bonded to aspartic acid (first step); preparing a conjugate in which octylamine (OCT) is bonded to aspartic acid (second step); and binding the conjugate of the first step and the conjugate of the second step (third step).

### Advantageous Effects

According to the present disclosure, it was found that a polypeptide polymer, which is obtained by binding a conjugate in which diethylenetriamine (DET) is bonded to aspartic acid; and a conjugate in which octylamine (OCT) is bonded to aspartic acid in a specific order, exhibits superior mRNA transcription efficiency to a polymer bonded in a different order, such that it may be useful as the polypeptide polymer, a method for preparing the same, or a composition for delivering RNA including the same.

### BRIEF DESCRIPTION OF DRAWINGS

FIG. 1 shows synthesis pathways of Fmoc-L-Asp(OCT)-OH (A); and a sequence-controlled polypeptide which has diethylenetriamine (hereinafter referred to as DET) and octylamine (hereinafter referred to as OCT) moieties.

FIG. 2 shows NMR analysis results of Fmoc-L-Asp(OCT)-OBn (Experimental Example 2-2-3) (Top for ¹H NMR, bottom for ¹³C NMR data).

FIG. 3 shows NMR analysis results of Fmoc-L-Asp(OCT)-OH (Experimental Example 2-2-4) (Top for ¹H NMR, bottom for ¹³C NMR data).

FIG. 4 shows results of predicted and measured mass spectral analysis of polypeptides. A; DODODODO, B; DDOODDOO, C; DDDDOOOO, D; 12 mer DDOO(=DDOODDOODDOO), E; 16 mer DDOO(=DDOODDOODDOODDOO), F; 20 mer DDOO(=DDOODDOODDOODDOODDOO).

FIG. 5 shows HPLC spectral analysis results of polypeptides. A; DODODODO, B; DDOODDOO, C; 12 mer DDOO, D; 16 mer DDOO, E; 20 mer DDOO.

FIG. 6 shows results of HPLC spectral (left) and mass spectral (right) analysis of polypeptides. A; 20 mer DDO(O), B; 20 mer (D)DOO, (C) 20 mer (D)(D)OO, and D; 20 mer (D)D(O)O. In the experimental group, D or O in parentheses has a D-form, and D or O not in parentheses has an L-form. Parentheses are used for the sole purpose of indicating a D-form.

FIG. 7 shows structures and citations of polypeptides prepared in the present disclosure.

FIG. 8 shows a result of structural analysis for D/L isomers of DDOODDOO measured via GC-MS.

FIG. 9 shows a result (A) of analyzing a degree of protonation of an octameric polypeptide (pKa graph); a result (B) of analyzing light scattered intensity of an octameric polypeptide; a result (C) of analyzing light scattered intensity of 12, 16 and 20 mer DDOO polypeptides; a result (D) of analyzing circular dichroism (CD) spectroscopic spectra of an octameric polypeptide and 20 mer DDOO polypeptide; and a result (E) of analyzing a molecular structure of a DDOODDOO polypeptide.

FIG. 10 shows structures of DDDDOOOO (A), DODODODO (B), and DDDDDDDD (C) predicted by all-atom molecular dynamics (AAMD) simulations. The backbone is shown in red, a DET group in blue, an OCT group in gray, and a β-turn-like structure in purple.

FIG. 11 shows results of agarose gel electrophoresis analysis of polyplexes prepared according to an N/P ratio between sequence-controlled polypeptides and FLuc-mRNAs. The definition of the N/P ratio is a molar ratio of amino groups in a DET unit and phosphate groups in the mRNA.

FIG. 12 shows results of luminescence intensity analysis of A549 cells transfected with FLuc-mRNA for 24 hours. Specifically, FIG. 12 shows a result (A) of luminescence intensity analysis of cells transfected with polyplexes loaded with mRNA prepared from an octameric polypeptide in various N/P ratios; a result (B) of luminescence intensity analysis of polyplex-transfected cells transfected with 12, 16 and 20 mer DDOO(N/P=5) polypeptides; a result (C) of analyzing cellular uptake of Cy5-mRNA-loaded polyplexes (N/P=5,500 ng mRNA/well) in the cell (FIG. 12B); a result (D) of agarose gel electrophoresis assay of IVT mRNA of polyplexes (N/P=5) cultured in 10% FBS at 37° C. for 1 hour; and a result (E) of luminescence intensity analysis of cells transfected with FLuc-mRNA in 100 ng mRNA/well for 24 hours. All outcomes are expressed as mean±standard deviations (n=4).

FIG. 13 is the original data of FIG. 12C, showing results of analyzing the cellular uptake of polyplexes (N/P=5,500 ng mRNA/well) loaded with Cy5-mRNA in A549 cells cultured for 4 hours. A control group (polyplex untreated group) is shown in blue (★), and the polyplex treated group in red (☆).

FIG. 14 shows results of analyzing in vitro gene editing efficacy using HEK293-loxP-GFP-RFP (Neo) cells exhibiting red fluorescence after LoxPs are cleaved by Cre recombinase. Specifically, FIG. 14 shows a schematic diagram (A) of HEK293 cells expressing a LoxP-GFP-stop-LoxP-RFP cassette, an image (B) of CLSM observation of HEK293-loxP-GFP-RFF treated with 20 mer DDOO/Cre-mRNA for 48 hours (100 ng mRNA/well), and a result (C) of analyzing Cre recombinase activity through the number of GFP or/and RFP-expressing cells and their fluorescence intensity.

FIG. 15 shows results of analyzing in vitro gene editing efficacy using HEK293-loxP-GFP-RFP (Neo) cells exhibiting red fluorescence after LoxPs are cleaved by Cre recombinase. Specifically, FIG. 15 shows (A) cells untreated with a sample, (B) cells treated with 20 mer DDOO and Cre-mRNA for 48 hours, and (C) cells treated with lipofectamine 3000 (positive control group) for 48 hours.

FIG. 16 shows results (A)-(D) of analyzing isothermal titration calorimetry (ITC) profiles of 8 mer DDOO/ssRNA (A), 12 mer DDOO/ssRNA (B), 16 mer DDOO/ssRNA (C), and 20 mer DDOO/ssRNA (D). In (A) to (D) of FIG. 16, the top shows the raw ITC data, and the bottom shows the plot of heat flow in accordance with a molar ratio between polypeptides and ssRNAs. (E) of FIG. 16 shows results of analyzing AAMD simulation between DDOO polymers and 20 mer RNAs, and (F) of FIG. 16 shows optical images that observed polyplexes containing 20 nt ssRNA (N/P=5).

### BEST MODE FOR CARRYING OUT THE INVENTION

Hereinafter, the present disclosure will be described in detail.

The present disclosure provides a polypeptide polymer represented by the following Chemical Formula 1:

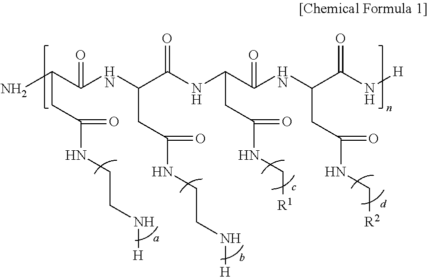

In the Chemical Formula 1, n is one of 1 to 20, a and b may be the same or different and are each independently one of 1 to 5, c and d may be the same or different and are each independently one of 1 to 20, and R¹ and R² may be the same or different and are each independently hydrogen or (C4-C12)cycloalkyl,

preferably, n is one of 1 to 5, a and b may be the same or different and are each independently one of 1 to 3, c and d may be the same or different and are each independently one of 5 to 10, and R¹ and R² may be the same or different and are each independently hydrogen or (C4-C8)cycloalkyl, and

more preferably, n may be each one of 1 to 5, a and b may be each one of 1 to 3, c and d may be each one of 5 to 10, and R¹ and R² may be each hydrogen or (C4-C8)cycloalkyl.

The polymer may be in a form in which diethylenetriamine (DET) or octylamine (OCT) is bonded to aspartic acid, wherein the form in which diethylenetriamine (DET) or octylamine (OCT) is bonded to aspartic acid may have an L-form or D-form.

The polypeptide polymer may increase RNA transcription efficiency, and the RNA may be one or more selected from the group consisting of mRNA, siRNA, antisense oligonucleotide (ASO), and guide RNA (gRNA), but is not limited thereto.

The mRNA may be an in vitro transcribed (IVT) mRNA.

In addition, the present disclosure provides a composition for delivering RNA, including the polypeptide polymer.

In addition, the present disclosure provides a reagent composition for promoting RNA transcription, including the polypeptide polymer.

In addition, the present disclosure provides a method for preparing a polypeptide polymer represented by the Chemical Formula 1, the method including: preparing a conjugate in which diethylenetriamine (DET) is bonded to aspartic acid (first step); preparing a conjugate in which octylamine (OCT) is bonded to aspartic acid (second step); and binding the conjugate of the first step and the conjugate of the second step (third step).

### MODES FOR CARRYING OUT THE INVENTION

Hereinafter, to help understanding of the present disclosure, example embodiments will be described in detail. However, the following example embodiments are merely illustrative of the content of the present disclosure, and the scope of the present disclosure is not limited to the following example embodiments. The example embodiments of the present disclosure are provided to more completely explain the present disclosure to those with ordinary skill in the art.

### [Experiment Example 1] Preparing for Experiments

Dulbecco's modified Eagle's medium (DMEM), phosphate-buffered saline (PBS), fetal bovine serum (FBS), trypsin-EDTA, and ultrapure agarose were purchased from ThermoFisher Scientific (Waltham, MA, USA). HEPES buffer (1M, pH 7.3) was purchased from Amresco (Solon, OH, USA).

A549 cells, human lung cancer cells, were purchased from ATCC (Manassas, VA, USA). HEK293-loxP-GFP-RFP (Neo) cells were purchased from GenTarget Inc (Manassas, VA, USA). Firefly luciferase-coded mRNAs L-7202 (FLUC-mRNA) and Cre-recombinase-coded mRNAs (Cre-mRNA, L-7211) were purchased from TriLink Biotechnologies (San Diego, CA, USA). Organic solvents and reagents were purchased and used without further purification unless otherwise mentioned. Single-stranded RNA (ssRNA, 5′-AUGA GGACG CCAAG AACAU-3′) was used by synthesizing from Macrogen (Seoul, Korea). Fluorescently labeled FLUC-mRNA was prepared by attaching a fluorescent dye using a Label IT Cy5 labeling kit (Mirus Bio Corporation, Madison, WI, USA). The melting point (m.p) was measured by Leica Galen III microscope and expressed in degrees Celsius (C). Infrared (IR) spectra were recorded using Nicole IR100 with NaCl crystals as a film or Nico IR100 with Nujol as a solvent. ¹H and ¹³C NMR spectra were recorded using Bruker Advance NEO 400 MHz with Prodigy CPBBBO BB-H&F z-gradient cryo-probe spectrometer at room temperature. Chemical shifts were expressed in ppm using the reported shifts and solvent signals (1H/13C: deuterated chloroform CDCl₃ 7.26/77.2 ppm, dimethyl sulfoxide DMSO-d62.49/39.5 ppm) as an internal standard. The coupling constant (J) is expressed in hertz and abbreviated as follows to describe multiplicity: s; singlet, d; doublet, t; triplet, q; quintet, sext; sextet, m; multiplet, br; broad. Mass spectra were recorded using electrospray ionization (ESI) by means of Thermo Q Exactive Focus or Waters ZQ 400 spectrometer.

### [Experimental Example 2] Synthesis of Polypeptides

### 2-1. Crude Purification of Reactions

The reaction of monomers was monitored by a thin layer chromatograph (TLC) using a pre-coated aluminum-backed plate (0.2 mm silica gel 60 F254, Merck®) and visualized with UV light. Purification of compounds was performed using silica gel column chromatography (Chromagel 60A SdS.C.C. 70-200 μm) along with a solvent mixture whose polarity increases as an eluent.

### 2-2. Synthesis of Fmoc-L-Asp(OCT)-OH

### 2-2-1. Preparation of Fmoc-L-Asp(OtBu)-OBn

Fmoc-L-Asp(OtBu) (20.0 g, 48.6 mmol) was dissolved in 70 mL of dimethylformamide (DMF). Afterwards, well-powdered KOH (2.72 g, 48.6 mmol) was added, and benzyl chloride (16.8 ml, 145.8 mmol) was added. The solution was stirred under argon atmosphere at room temperature for 24 hours. Subsequently, the solution was added to 300 mL of 2M aqueous HCl, the sediment was filtered under vacuum, washed three times with water, and washed three times again with hexane to obtain a white solid form of Fmoc-L-Asp(OtBu)-OBn (20.0 g, 82%). Spectral characteristics were consistent with those reported in the document. The chemical formula of the Fmoc-L-Asp(OtBu)-OBn is as follows, and the IUPAC name is 1-benzyl 4-(tert-butyl)(((9H-fluoren-9-yl)methoxy)carbonyl)-L-aspartate.

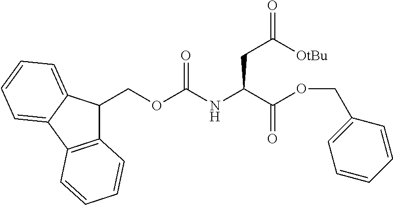 [SMILES: CC(C)(C)OC(=O)C[C@H](NC(=O)OCC1c2ccccc2-c2ccccc21)C(=O)OCc1ccccc1]

### 2-2-2. Preparation of Fmoc-L-Asp(OH)-OBn

Fmoc-L-Asp(OtBu)-OBn (10.0 g, 22.4 mmol) was suspended in dichloromethane (DCM, 15 mL), and trifluoroacetic acid (TFA, 15 mL) was added to the mixture. The solution was stirred at room temperature for 8 hours and concentrated under reduced pressure. Trace amounts of TFA were removed by co-evaporation with DCM (×3) to produce Fmoc-Asp(OH)-OBN (9.98 g) in the form of brown oil. Spectral characteristics were consistent with those reported in the document. The chemical formula of the Fmoc-L-Asp(OH)-OBn is as follows, and the IUPAC name is(S)-3-((((9H-fluoren-9-yl)methoxy)carbonyl)amino)-4-(benzyloxy)-L-4-oxobutanoic acid.

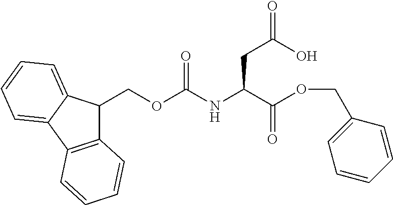 [SMILES: O=C(O)C[C@H](NC(=O)OCC1c2ccccc2-c2ccccc21)C(=O)OCc1ccccc1]

### 2-2-3. Preparation of Fmoc-L-Asp(OCT)-OBn

Fmoc-L-Asp(OH)-OBn (9.98 g, 22.4 mmol) was dissolved in DCM (100 mL), and the solution was cooled in an ice bath under an argon atmosphere. Afterwards, hexafluorophosphate azabenzotriazole tetramethyl uranium (HATU, 8.52 g, 22.4 mmol) was added to the solution. N,N-diisopropylethylamine (DIEA, 7.8 mL, 44.8 mmol) was also added to the solution. The mixture was stirred at 0° C. for 30 minutes, and a solution of 1-octylamine (3.7 mL, 22.4 mmol) in DCM (30 mL) was added. The solution was stirred at room temperature for 1 hour, transferred to a separatory funnel, washed twice with 2M aqueous HCl solution and 10% water-soluble NaHCO₃, and dried with anhydrous Na₂SO₄. The solution was filtered and evaporated under reduced pressure. The crude mixture was purified by silica gel column chromatography using a mixture of DCM-EtOAc (0~15%) as an eluent, and the white solid form of Fmoc-L-Asp(OCT)-OBn (11.5 g, 92%) was obtained. The chemical formula of the Fmoc-L-Asp(OCT)-OBn is as follows, the IUPAC name is benzyl N²-(((9H-fluoren-9-yl)methoxy)carbonyl)-N⁴-octyl-L-asparaginate, and the NMR analysis results are as follows.

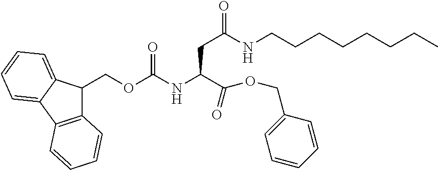 [SMILES: CCCCCCCCNC(=O)C[C@H](NC(=O)OCC1c2ccccc2-c2ccccc21)C(=O)OCc1ccccc1]

m.p.: 121-123° C.

IR (CDCl₃, υ in cm⁻¹): 3299, 3066, 3033, 2923, 2852, 1755, 1684, 1645, 1547, 1450, 1294.

¹H NMR (400 MHz, CDCl₃) δ 7.70 (d, J=7.5 Hz, 2H), 7.53 (d, J=7.4 Hz, 2H), 7.33 (t, J=7.5 Hz, 2H), 7.31-7.19 (m, 7H), 6.20 (d, J=8.0 Hz, NH), 5.73-5.63 (m, NH), 5.20-5.09 (m, 2H), 4.62 (s, 1H), 4.57 (dd, J=8.2, 4.1 Hz, 1H), 4.38-4.30 (m, 1H), 4.27-4.20 (m, 1H), 4.14 (t, J=7.1 Hz, 1H), 3.11 (dd, J=13.0, 6.4 Hz, 1H), 2.85 (dd, J=15.6, 4.0 Hz, 1H), 2.66 (dd, J=15.6, 4.0 Hz, 1H), 1.34 (d, J=18.3 Hz, 2H), 1.20 (s, 10H), 0.83 (t, J=6.5 Hz, 3H).

¹³C NMR (100 MHz, CDCl₃) δ 171.1 (C), 169.7 (C), 156.4 (C), 144.0 (C), 143.8 (C), 141.3 (C), 141.1 (C), 135.5 (C), 128.6 (CH), 128.6 (CH), 128.4 (CH), 128.2 (CH), 127.8 (2CH), 127.6 (2CH), 127.2 (CH), 127.0 (CH), 125.3 (CH), 125.3 (CH), 120.0 (CH), 67.5 (CH₂), 67.4 (CH₂), 65.3 (CH₂), 51.2 (CH), 47.2 (CH), 39.8 (CH₂), 37.7 (CH₂), 31.9 (CH₂), 29.6 (CH₂), 29.3 (CH₂), 27.0 (CH₂), 22.7 (CH₂), 14.2 (CH₃).

HRMS (ESI+): Calculated for C₃₄H₄ON₂NaO₅+[M+H]⁺ 579.2829, found 579.2824.

$${[ a ]}_{D}^{20}$$

### +13.7 (c 0.8, CHCl₃).

### 2-2-4. Preparation of Fmoc-L-Asp(OCT)-OH

Fmoc-L-Asp(OCT)-OBn (3.87 g, 8.29 mmol) was dissolved in a mixture in which EtOAc (30 mL) and MeOH (10 mL) are mixed, and the solution was transferred to a pressure vessel filled with 5% Pd/C (112 mg, 0.05 mmol). A hydrogen cylinder was connected to a pressure vessel, and the reaction mixture was stirred at a pressure of 4 Pa of H₂ for 18 hours. Subsequently, the reaction mixture was diluted in CHCl₃ (200 mL), refluxed until the product was dissolved, and the hot solution was filtered through a cotton pad to obtain a white solid form of Fmoc-L-Asp(OCT)-OH (2.2 g, 57%). The chemical formula of the Fmoc-L-Asp(OCT)-OH is as follows, the IUPAC name is benzyl N²-(((9H-fluoren-9-yl)methoxy)carbonyl)-N⁴-octyl-L-asparagine, and the NMR analysis results are as follows.

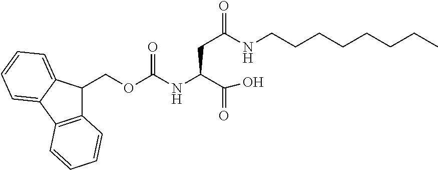 [SMILES: CCCCCCCCNC(=O)C[C@H](NC(=O)OCC1c2ccccc2-c2ccccc21)C(=O)O]

m.p.: 174-176° C.

IR (nujol, υ in cm⁻¹): 3338, 2929, 2852, 1755, 1690, 1599, 1534, 1463, 1372.

¹H NMR (400 MHz, DMSO-d₆) δ 7.89 (d, J=7.5 Hz, 2H), 7.71 (d, J=7.4 Hz, 2H), 7.42 (t, J=7.4 Hz, 2H), 7.32 (t, J=7.3 Hz, 2H), 4.39-4.31 (m, 1H), 4.30-4.16 (m, 3H), 3.02 (dd, J=12.6, 6.5 Hz, 2H), 2.58 (dd, J=15.0, 5.1 Hz, 1H), 2.58 (dd, J=15.0, 5.1 Hz, 1H), 1.42-1.30 (m, J=6.7 Hz, 2H), 1.20 (s, 10H), 0.83 (t, J=6.7 Hz, 3H).

¹³C NMR (101 MHz, DMSO-d₆) δ 173.1 (C), 168.9 (C), 155.7 (C), 143.8 (2C), 140.7 (2C), 127.6 (2CH), 127.1 (2CH), 125.2 (2CH), 120.1 (2CH), 65.7 (CH₂), 51.0 (CH), 46.6 (CH), 38.6 (CH₂), 37.3 (CH₂), 31.2 (CH₂), 30.1 (CH₂), 28.7 (CH₂), 28.6 (CH₂), 26.4 (CH₂), 22.1 (CH₂), 13.9 (CH₃).

HRMS (ESI+): Calculated for C₂₇H₃₄N₂NaO₅⁺ [M+Na]⁺ 489.2360, found 489.2359.

$${[ a ]}_{D}^{20}$$

### −14.1 (c 0.9 DMSO)

### 2-3. Synthesis of Fmoc-D-Asp(OCT)-OH

### 2-3-1. Preparation of Fmoc-D-Asp(OtBu)-OBn

Fmoc-D-Asp(OtBu)-OBn was prepared in the same method as the method of preparing the Fmoc-L-Asp(OtBu)-OBn (Experimental Example 2-2-1). The chemical formula of the Fmoc-D-Asp(OtBu)-OBn is as follows, and the IUPAC name is 1-benzyl 4-(tert-butyl)(((9H-fluoren-9-yl)methoxy)carbonyl)-D-aspartate.

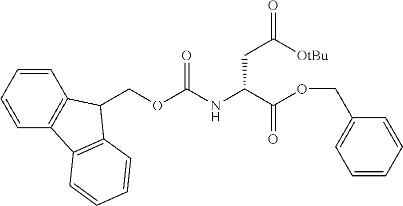 [SMILES: CC(C)(C)OC(=O)C[C@@H](NC(=O)OCC1c2ccccc2-c2ccccc21)C(=O)OCc1ccccc1]

### 2-3-2. Preparation of Fmoc-D-Asp(OH)-OBn

Fmoc-D-Asp(OH)-OBn was prepared in the same method as the method of preparing the Fmoc-L-Asp(OH)-OBn (Experimental Example 2-2-2). The chemical formula of the Fmoc-D-Asp(OH)-OBn is as follows, and the IUPAC name is(S)-3-((((9H-fluoren-9-yl)methoxy)carbonyl)amino)-4-(benzyloxy)-D-4-oxobutanoic acid.

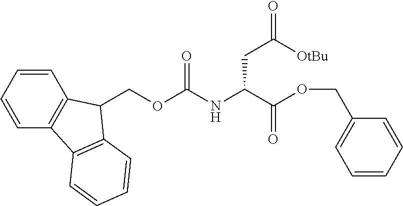 [SMILES: CC(C)(C)OC(=O)C[C@@H](NC(=O)OCC1c2ccccc2-c2ccccc21)C(=O)OCc1ccccc1]

### 2-3-3. Preparation of Fmoc-D-Asp(OCT)-OBn

Fmoc-D-Asp(OCT)-OBn was prepared in the same method as the method of preparing the Fmoc-L-Asp(OCT)-OBn (Experimental Example 2-2-3). The chemical formula of the Fmoc-D-Asp(OCT)-OBn is as follows, and the IUPAC name is benzyl N²-(((9H-fluoren-9-yl)methoxy)carbonyl)-N⁴-octyl-D-asparaginate.

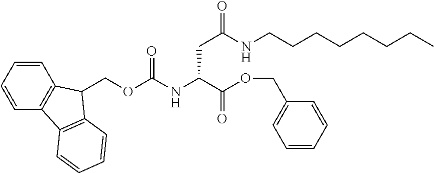 [SMILES: CCCCCCCCNC(=O)C[C@@H](NC(=O)OCC1c2ccccc2-c2ccccc21)C(=O)OCc1ccccc1]

$${[ a ]}_{D}^{20}$$

### −14.1 (c 0.8, CHCl₃).

### 2-3-4. Preparation of Fmoc-D-Asp(OCT)-OH

Fmoc-D-ASP (OCT)-OH was prepared in the same method as the method of preparing the Fmoc-L-Asp(OCT)-OH (Experimental Example 2-2-4). The chemical formula of the Fmoc-D-Asp(OCT)-OH is as follows, and the IUPAC name is benzyl N²-(((9H-fluoren-9-yl)methoxy)carbonyl)-N⁴-octyl-D-asparagine.

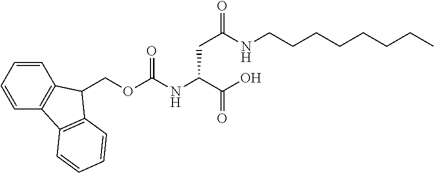 [SMILES: CCCCCCCCNC(=O)C[C@@H](NC(=O)OCC1c2ccccc2-c2ccccc21)C(=O)O]

$${[ a ]}_{D}^{20}$$

### +7.81 (c 1.5, DMSO).

2-4. Synthesis of polypeptides

The polypeptides were synthesized as follows. Rink Amide AM resin LL (100-200 mesh), a special type of resin with low substitution, consists of a modified Rink Amide Linker introduced to aminomethyl polystyrene (Sigma Aldrich, Nobabichem, 855120) at 0.1 mmol/g, and the resin was swollen with 3 mL of DMF in a poly-prep chromatography column (Bio-Rad) for 30 minutes and then subjected to cycles of Fmoc-α-amino acid coupling. A single cycle of the coupling consists of a two-stage processing step that includes the removal of an FMOC protecting group and subsequent coupling of amino acids. In each cycle, the Fmoc group was removed with 3 mL of 20% piperidine in DMF under the stirring condition for 15 minutes, and the resin was washed three times with 3 mL of DMF in a poly-prep column. The resin was then bonded with a mixture of Fmoc-L-Asp(OtBu) (4 equiv), Fmoc-D-Asp(OtBu) (4 equiv), Fmoc-L-Asp(OCT)-OH (3 equiv), or Fmoc-D-Asp(OCT)-OH (3 equiv) along with HATU (the same as Fmoc-amino acids) and DIEA (6 equiv). The resin including the mixture in the column was shaken for 30 minutes for the coupling process, and the resin was washed three times with 3 mL of DMF. Bound to the resin respectively after 8, 12, 16, and 20 cycles, the octameric, dodecameric, hexadecameric, and icosameric polypeptides were treated with diethylenetriamine (DET), and the resin was stirred in a 50% DET solution (5 mL) at 4° C. for 48 hours, followed by replacement with a new DET solution four times. The resulting peptide analogues were isolated from the resin with 2 mL of TFA cocktail consisting of TFA (90%), DCM (5%), triisopropylsilane (2.5%), and water (2.5%). Peptide analogues were separated by precipitation in diethyl ether and centrifugation, and mass was measured by mass spectrometry (Thermo Q Exactive Focus or Waters ZQ 4000).

### 2-5. Analysis of High-Purity Polypeptides

The purity of the polypeptide was analyzed via HPLC (KNAUER, Germany, comprised of Smart time manager 5000 with degasser 10 ml; LPG (E43OV2, 104107), Smart time pump 1000 including 10 ml pump head titanium inlays (EA4300V1, 95270), and UV detector 2500 (E4310, 103886)), and pentafluorophenyl (PFP) columns (Phenomenex, US, PNO 00F-4447-EQ, Luna 3u PFP (2) 100Å, 150×4.60 mm, SNO 438270-1) were used. [A=H₂O+0.1% TFA, B=ACN+0.1% TFA] gradient; 0~5 minutes (5% B), ~25 minutes (95% B), flow rate of 2 ml/min.

### 2-6. Purification of Polypeptides

Purification of polypeptides was performed by HPLC (KNAUR, Germany) using a 3 μm Evosphere PFP semi-prep column (Fortis, UK, size 250×10 mm, SN P01232305-2, PN EVOPFP-100903). [A=H₂O+0.1% TFA, B=ACN+0.1% TFA] gradient; 0~5 minutes (5% B), ~25 minutes (95% B), flow rate of 5.5 ml/min.

### [Experiment Example 3] Analysis of DDOODDOO Enantiomers

Optical purity of DDOODDOO was analyzed via gas chromatography-mass spectrometry (GC-MS) by HiPep Laboratories (Kyoto, Japan) using an enantiomer labeling method. The polypeptide was hydrolyzed in 6N HCl in the presence of D₂O, and the residue was acylated using trifluoroacetic anhydride or pentafluoropropionic anhydride. The dissolved residues were injected into the GC-MS. The purity of enantiomers (% D enantiomer) was calculated by area of D enantiomer/(area of D enantiomer+area of L enantiomer)×100.

### [Experiment Example 4] Analysis of a Degree of Protonation

Octameric polypeptide (20 mg) was dissolved in 50 mL of 0.005M HCl containing 150 mM NaCl and titrated with 0.025 M NaOH containing 150 mM NaCl at room temperature. An automatic titrator (COM-A19, Hiranuma, Kyoto, Japan) was used for titration. After the pH value in the solution was stabilized, a titrant was added in an automated amount of 0.05~0.5 mL. From the obtained titration curve, the relationship between pH and the degree of protonation of polycations (a) was calculated.

### [Experimental Example 5] Light Scattered Analysis

For the light scattered intensity of the polypeptide, the static light scattering was measured at 25° C. and a detection angle of 173° using Zetasizer Pro (Malvern Panalytical, Worcestershire, UK) equipped with a He—Ne laser (2=633 nm). Samples in 10 mM HEPES buffer (pH 7.3) were analyzed using a low-volume quartz cuvette (ZEN2112, Malvern Panalytic).

### [Experimental Example 6] Circular Dichroism Spectroscopic Analysis

The secondary structure of peptides was measured by circular dichroism (CD) spectroscopy (J-815, Jasco, Japan) at room temperature. Polypeptides at a concentration of 0.5 mg/mL in 10 mM sodium phosphate buffer (pH 7.0) were analyzed using a cuvette (path length: 10 mm, Hellma, Mullheim, Germany).

### [Experimental Example 7] Preparation of FLuc-mRNA-Loaded Polyplexes and Characteristic Analysis

Octameric polypeptide was first dissolved in 0.01 M HCl at a concentration of 4 mg/mL and stirred at 4° C. for 1 hour to replace the counterion from trifluoroacetate to chloride. Afterwards, 10 mM HEPES buffer (pH 7.3) was added to the polypeptide solution to achieve a concentration of 2 mg/mL. The octameric polypeptide was further diluted in 10 mM HEPES buffer (pH 7.3) and then mixed with FLuc-mRNA solution (100 ng/μL mRNA in 10 mM HEPES buffer, pH 7.3) to prepare an mRNA-loaded polyplex (20 ng/μL mRNA) in a desired molar ratio of the amino group in the DET moiety to the phosphate group in the FLuc-mRNA at a desired N/P ratio. 12 mer, 16 mer, and 20 mer DDOO polypeptides were dialyzed for 0.01M HCl to replace the counterions from acetate to chloride. Finally, the polypeptides were subjected to dialysis for deionized water and then lyophilized. For further experiments, the polypeptides were dissolved in 10 mM HEPES buffer (pH 7.3).

Polyplex size (cumulant diameter) and size distribution (polydispersity index (PDI) were measured by dynamic light scattering using a Zetasizer Pro (Red) equipped with a He—Ne laser (2=633 nm) at 25° C. and a detection angle of 173°. A low-capacity quartz cuvette (ZEN2112) was used for size measurement. Zeta-potential of polyplexes was measured with the same device at 25° C. using folded capillary cells (DTS1070, Malvern Panalytical). The zeta potential was calculated using electrophoretic mobility measured based on the Smoluchowski equation.

Optical microscopy (Axio Observer, Carl Zeiss, Germany) was used to analyze the macroscopic properties of polyplexes. 8 mer to 20 mer DDOO in 10 mM HEPES buffer (pH 7.3) was mixed with ssRNAs in a length of 20 nucleotides (100 ng/μL ssRNA in 10 mM HEPES buffer, pH 7.3) for 1 hour at N/P=5. Polyplexes (10 μL, 20 ng/μL ssRNA) were imaged at room temperature on an 8-well Lab-Tek chambered borosilicate cover glass (Nalge Nunc International, Rochester, NY).

### [Experimental Example 8] Gel Retardation Assay

Polypeptides and FLuc-mRNA were mixed in a desired N/P ratio as mentioned above. Polyplex solution was mixed with glycerol (final concentration of glycerol: 8 vol %, final amount of mRNA: 100 ng), and samples were subjected to electrophoresis in agarose gel (1.2 wt % agarose gel, 1×TBE buffer, 135 V, 15 minutes). The mRNA in the gel was stained with ethidium bromide and visualized using the WSE-5300 Printgraph CMOS (ATTO, Tokyo, Japan). To determine the stability of polyplexes for PBS containing 10% FBS (PBS/FBS), polyplexes (N/P=5, 100 ng, 10 μL) were mixed with PBS/FBS (90 μL) at 37° C. for 1 hour, the mixture was purified with RNeasy Mini Kit (Qiagen), and samples were subjected to electrophoresis in agarose gel (1.2 wt % agarose gel, 1×TBE buffer, 135 V, 15 minutes). The mRNA in the gel was then stained with ethidium bromide and visualized using the WSE-5300 Printgraph CMOS.

### [Experimental Example 9] mRNA Transfection with Polyplexes

A549 cells were seeded in DMEM medium containing 10% FBS (DMEM/FBS) in 96-well plates at a density of 8,000 cells/well, and the next day, polyplex solutions prepared from FLuc-mRNA were added to each well at varying N/P ratios or mRNA concentrations, followed by cell culture for 24 hours. The expression level of firefly luciferase was measured using the Luciferase Assay System (Promega, Madison, WI) by the photoluminescence intensity of the cell lysate, and the photoluminescence intensity was measured by the luminescent microplate reader (Mithras LB 963 Centro, Bertold Technologies GmbH & Co. KG, Bad Wildbad, Germany).

### [Experimental Example 10] Flow Cytometry

A549 cells were seeded in a 24-well plate at a density of 50,000 cells/well in DMEM/FBS, and the medium was replaced with a new DMEM/FBS the next day. Subsequently, a polyplex solution (N/P=5) including Cy5-labeled FLuc-mRNA (Cy5-mRNA) was added to each well (500 ng mRNA/well). After 4 hours of culture, the medium was removed, and the cells were washed twice with 1 mL of PBS. Cells were treated with trypsin-EDTA solution for 1 minute and suspended in PBS. The fluorescence intensity of cell-derived Cy5-mRNA was measured using CytoFLEX (Beckman Coolter, CA, USA). The cells were excited with a 638 nm laser. The emitted fluorescence was detected using an APC channel with a 660/10 BP filter.

### [Experiment Example 11] Measurement by an Isothermal Titration Calorimeter

Measurement by an isothermal titration calorimeter (hereinafter referred to as ITC) was performed as follows. ssRNA (39.6 μM) in a nucleotide length of 20 and DDOO polypeptides (396 μM) were dissolved in 10 mM HEPES buffer (pH 7.3). Heat released during polyplex complexation between two samples was measured using MicroCal Auto-iTC200 (Malvern Panalytical, USA). Polypeptide samples were injected into RNA solution for 4 seconds at intervals of 150 seconds at 25° C. To calculate the enthalpy and entropy for complexation between RNA and peptides, a total of 19 drops were injected. Data were analyzed using MicroCal Origin 7.0 software.

### [Experimental Example 12] MD Simulation

Cationic polypeptides were parameterized using AmberTools19's Antechamber package for atomistic MD simulations (AAMD) using the allocated partial charge using the Gasteiger method. AAMD simulations were performed by Amber ff14SB in GROMACS 2021.4. To determine the polypeptide structure, the cationic polypeptide was first immersed in a simple point charge (SPC) water box, and chloride counterions were added therein to neutralize the charge of the system. The system was equilibrated for 100 ps in the NVT ensemble at 300 K using a V-scale thermostat with minimized energy. The Parrinello-Rahman barostat [29] was then used to achieve an NPT equilibrium of 100 ps at 1.0 bar. Afterwards, the Parrinello Rahman barostat was used to achieve NPT equilibrium of 100 ps at 1.0 bar. Finally, the system was simulated for 100 ns without limitation. For the complex simulation, RNA (sequence: AUGGA GGACG CCAAG AACAU) and cationic polypeptides were put into an SPC water box which is 2 nm away from the RNA, and the system was neutralized by adding sodium counterions. Afterwards, the system was minimized and, with equilibrium maintained as mentioned above, simulation was followed for 100 ns without limitation.

### [Experimental Example 13] Analysis of In Vitro Gene Editing Efficacy Using a Reporter System by mRNA Delivery

HEK293-loxP-GFP-RFP (Neo) cells were seeded in a 96-well optical bottom plate (165305, ThermoFisher Scientific) at a density of 8,000 cells/well in DMEM/FBS. HEK293-loxP-GFP-RFP cells include LoxP-GFP-stop-LoxP-RFP cassettes in the presence of the CMV promoter exhibiting red fluorescence after LoxPs are cleaved via Cre recombinase. The medium was replaced with a new DMEM/FBS the next day. Afterwards, a polyplex solution (N/P=5) containing Cre-mRNA was added to each well (100 ng mRNA/well). After 48 hours of culture, the cells were observed using a CLSM (ZEISS LSM 980, Carl Zeiss, Oberkochen, Germany) equipped with a 20× objective (Carl Zeiss). The amount of GFP or RFP positive cells and the fluorescence intensity in the images were analyzed using Image J.

### [Example 1] Synthesis of Sequence-Controlled Polypeptides

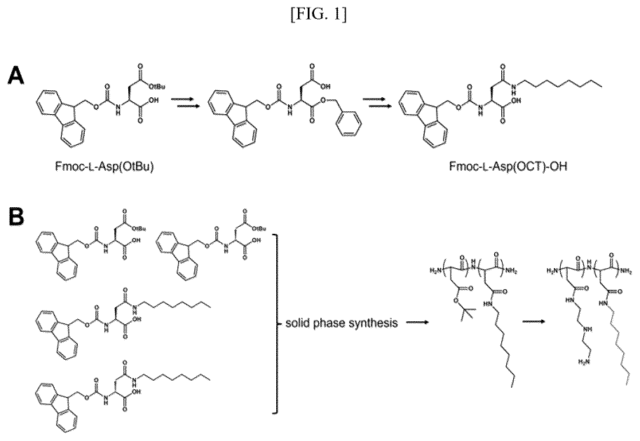

A series of sequence-controlled polypeptides, including DET and OCT moieties, were prepared via solid-phase peptide synthesis in which tert-butyl and Fmoc-protected aspartic acid (hereinafter referred to as Fmoc-L-Asp(OtBu) or Fmoc-D-Asp(OtBu)) and octyl modified aspartic acid (hereinafter referred to as Fmoc-L-Asp(OCT)-OH or Fmoc-D-Asp(OCT)-OH) are employed (FIG. 1). In the case of Fmoc-L-Asp(OtBu) synthesis, the carboxyl group in Fmoc-L-Asp(OtBu)-OBn was protected through a benzyl ester reaction to produce Fmoc-L-Asp(OtBu)-OBn. The Tert-butyl group was deprotected by trifluoroacetic acid treatment, and 1-octylamine was bound to Fmoc-L-Asp(OH)-OBn using HATU reagent. Subsequently, Fmoc-L-Asp(OCT)-OH was generated through deprotection of the benzyl ester with hydrogen (H₂) treated with Pd/C catalyst.

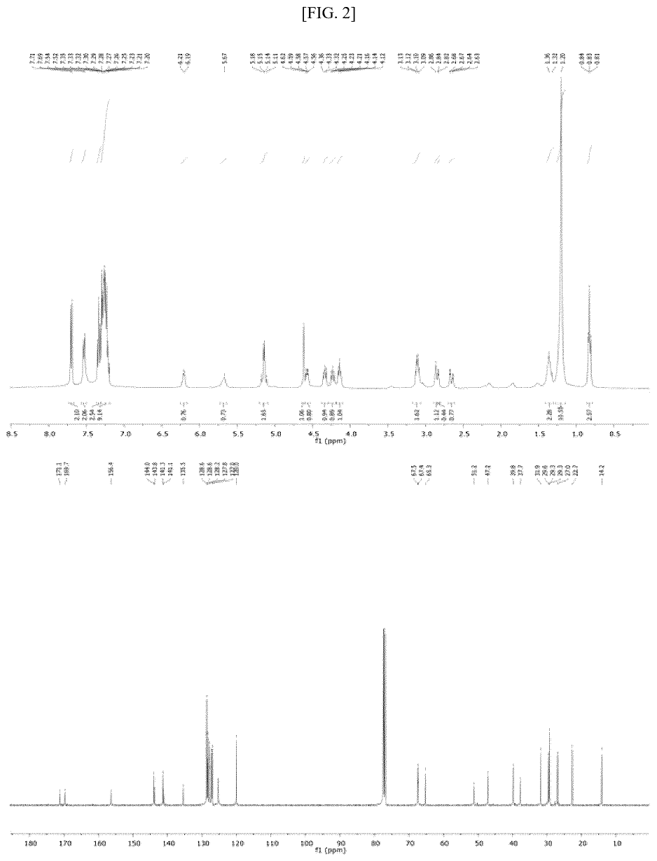

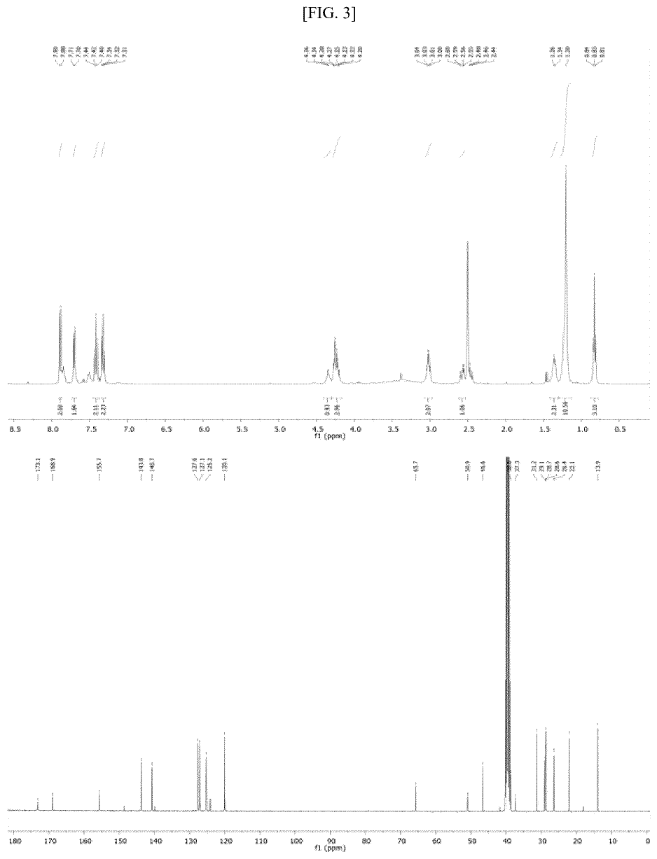

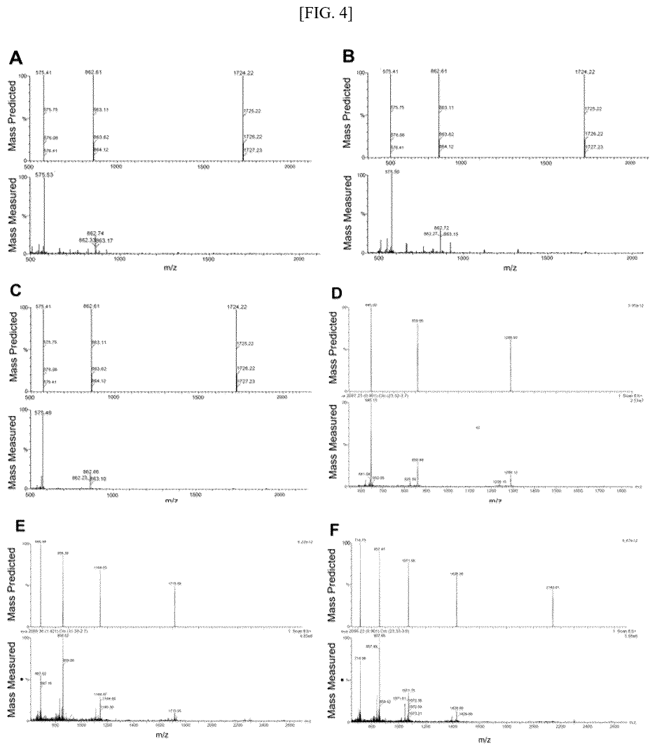

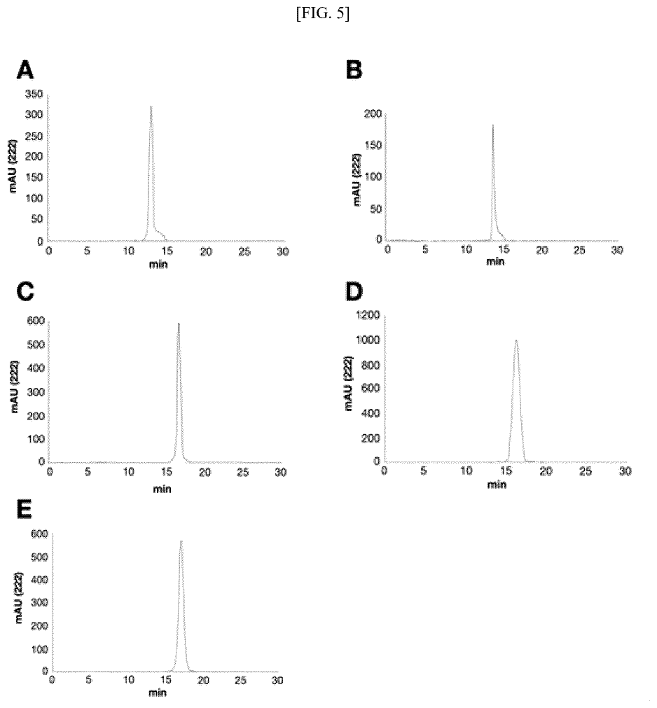

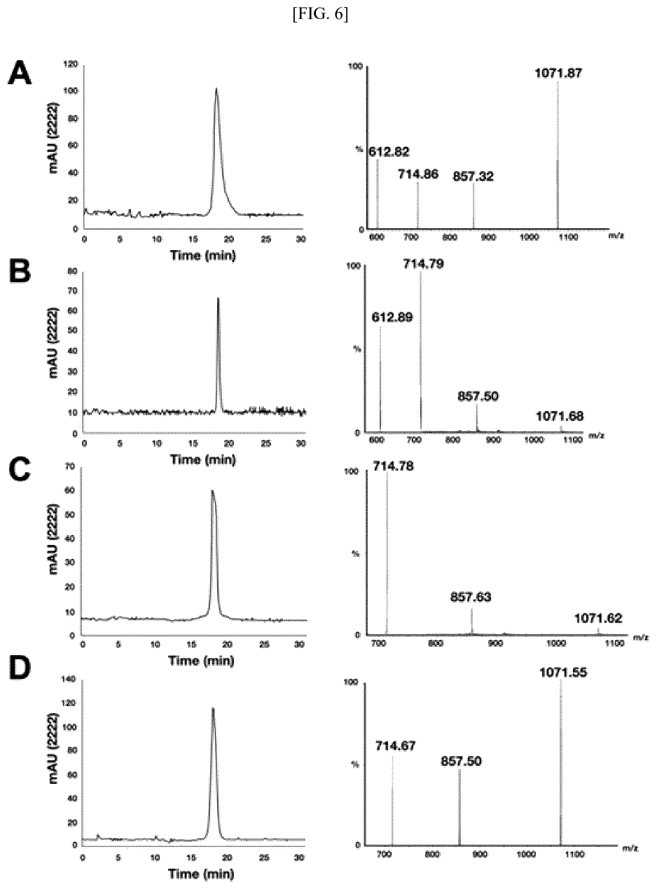

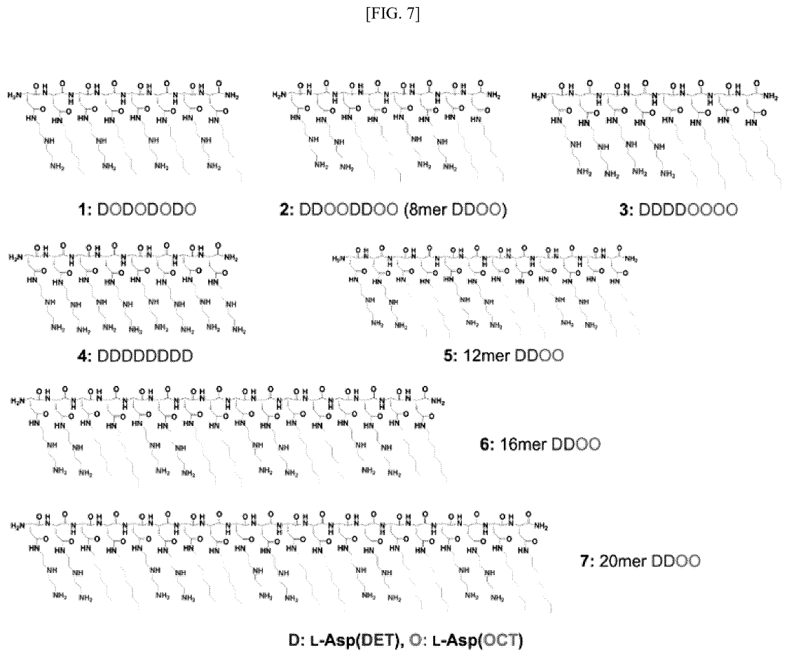

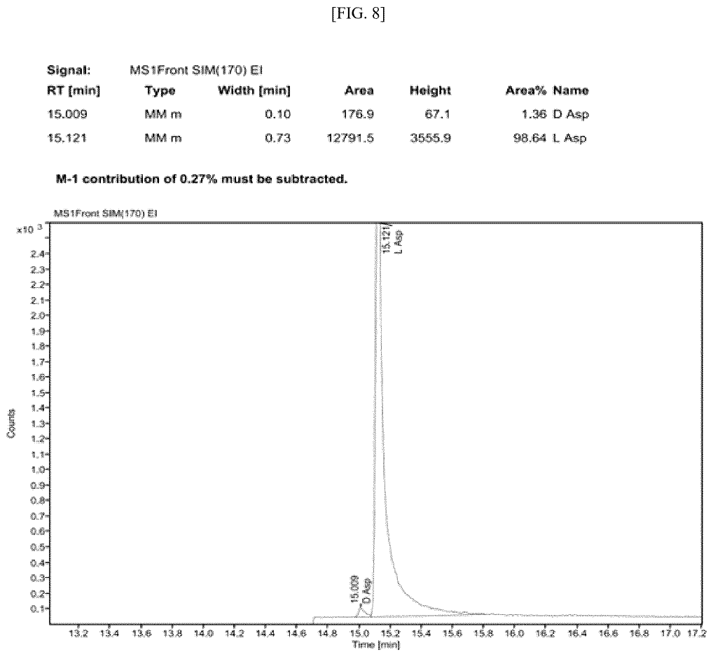

To determine whether Fmoc-L-Asp(OH)-OBn and Fmoc-L-Asp(OCT)-OH were successfully synthesized, ¹H NMR and ¹³C NMR NMR, IR spectroscopy, and mass spectrometry were performed (FIGS. 2 and 3). Fmoc-D-Asp(OCT)-OH was also synthesized via a similar protocol using Fmoc-D-Asp(OtBu). Various polypeptides were synthesized on resin to generate precursor polypeptides through repeated coupling cycles of Fmoc-L/D-Asp(OtBu) or Fmoc-L/D-Asp(OCT)-OH. Subsequently, the precursor polypeptide was reacted with an excess of DET to convert Asp(OtBu) to Asp(DET). The L-Asp(DET) and L-Asp(OCT) units were to be represented as D and O, respectively, while the D-Asp(DET) and D-Asp(OCT) units as (D) and (O), respectively. Two alternative copolypeptides (DODODO and DDOODDOO), one diblock copolypeptide (DDDDOOOO), and one homopolypeptide (DDDDDDD) were synthesized as octameric polypeptides. Three alternative copolypeptides (DDOO) having lengths (12 mer, 16 mer, 20 mer) longer than that of the alternative copolypeptide (DDOODDOO) were also prepared to analyze the effect of polypeptide length on mRNA complexation and in vitro transfection. The structure and abbreviations of each polypeptide are summarized in FIG. 7. Subsequently, D/L-20 mer DDOO was synthesized to prepare 20 mer DDO(O), (D)DOO, (D)(D)OO, and (D)D(O)O. Whether the synthesis of sequence-controlled polypeptides was successful was determined by mass spectrometry (FIG. 4 and FIG. 6). The purity of the polypeptide was analyzed using HPLC in a gradient solvent to identify single peaks of the product (FIG. 5 and FIG. 6). The optical purity of the octameric DDOODDOO was determined by GC-MS analysis pretreated by the enantiomer labeling method. As a result, DDOODDOO includes 98.6% L-form Asp, indicating that DET treatment to precursor polypeptides on the resin did not interfere with optical purity (FIG. 8).

### [Example 2] Analysis of a Degree of Protonation and Critical Association Concentration of Polypeptides

### 2-1. Analysis of a Degree of Protonation

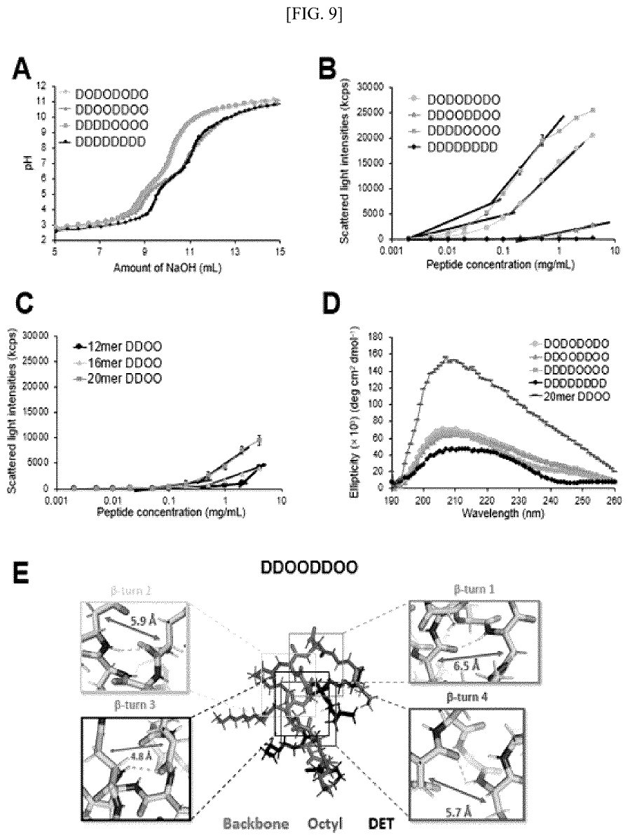

First, automatic potentiometric titration was used to analyze the degree of protonation of amino groups in the side chains of octameric polypeptides. Three octameric copolypeptides and one homopolypeptide used as the control group were dissolved in an acidic buffer (pH 2) in the presence of 150 mM NaCl, and the pH of the solution was measured by adding NaOH (FIG. 9A). As a result, the three copolypeptides showed each different degree of protonation. DODODODO and DDDDOOOO showed a lower buffering capacity from pH 5 to pH 7 compared to DDOODDOO, identifying that the sequence arrangement of amino groups has a significant effect on the degree of protonation. In the case of DODODODO, it was found that the hydrophobic octyl moiety adjacent to the amino group may induce a low buffer capacity by preventing the inflow/outflow of cations. Diblock copolypeptide (DDDDOOOO) immediately aggregated in the aqueous solution and inhibited the protonation of amino groups. On the other hand, DDOODDOO exhibited a degree of protonation similar to that of homopolypeptide (DDDDDDDD), which is widely studied for having high buffering capacity and endosomal escape ability after cell internalization, thereby discovering that at least two amino moieties should be separated from hydrophobic moieties in order to promote cation inflow/outflow towards the polypeptide.

### 2-2. Analysis of Critical Association Concentration

The critical association concentration (hereinafter referred to as CAC) of peptides measured by static light scattering was analyzed. Various concentrations (0.001~4 mg/ml) of polypeptides were dissolved in 10 mM HEPES buffer (pH 7.3), and the CAC of each polypeptide was calculated after measuring the scattered light intensity of each sample. As a result, DDOODDOO (CAC=0.304 mg/mL) had a CAC of about 2~3 times higher than those of DODODODO (CAC=0.155 mg/mL) and DDDDOOOO (CAC=0.083 mg/mL) (FIG. 9B). Homopolypeptide (DDDDDDDD) used as the control group was well dissolved in buffer (pH 7.3), but no CAC appeared up to treatment concentration of 4 mg/mL. Through the low CAC of DODODODO and DDDDOOOO, it was found that the degree of protonation of the polypeptides is not efficient. On the other hand, alternative polypeptides with longer lengths (12 mer, 16 mer, and 20 mer DDOO) exhibited CAC similar to octameric DDOO, identifying that the length of the polypeptide had no effect on CAC (FIG. 9C). From the above results, it was found that the inherent structure in the buffer affects the inflow/outflow of cations or the intermolecular interaction of each polypeptide, such that the sequence arrangement of polypeptides has a significant effect on the pKa value and CAC (or water solubility).

### [Example 3] Conformational Analysis of Polypeptides

At a concentration of 0.5 mg/mL (pH 7.0), the form of the octameric polypeptide was analyzed via circular dichroism (CD) spectroscopy. As a result, the spectra of octameric polypeptides appeared in the form of a single wide curve at the upper peak of 205~210 nm, regardless of the sequence arrangement (FIG. 9D). The spectra were analyzed using CDSSTR software, and for all octameric polypeptides, those taking β-turn were used (β-turn is an irregular secondary structure of peptides including intramolecular hydrogen bonds between the first and third residues; and four consecutive amino acid residues in a distance of 7 Å between the α-carbons).

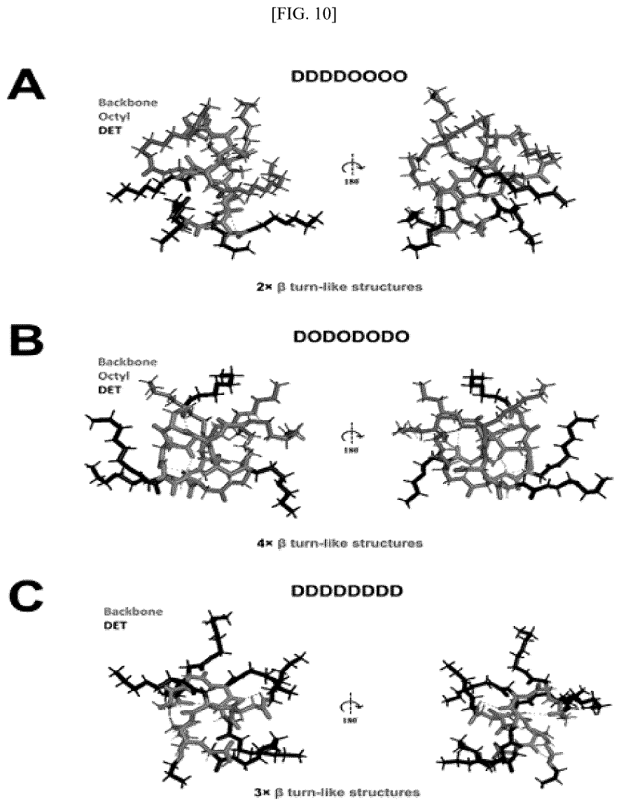

To determine how the polypeptide is able to adopt this irregular secondary structure, all-atom molecular dynamics simulation (AAMD) was performed. As a result, after equilibrating the polypeptide for 50 ns in a salt-neutralized water box, it was determined that all four polypeptides adopted specific β-turn characteristics (FIG. 9E and FIG. 10). In particular, structurally flexible DET and OCT side chains allowed the polypeptide backbone to form β-turn without excessive deformation. In addition, it was clearly observed that the DET side chains were well exposed to the outside in the three copolypeptides. Thereby, it was found that cations may freely flow in/out, with no limitation by its own secondary structure. Instead, in DDDDOOOO and DODODODO, the four OCT side chains were oriented in the same direction, inducing hydrophobic bonds among molecules. This supports the fact that the polypeptides (DDDDOOOO and DODODODO) had lower CAC levels than that of DDOODDOO. From the above results, it was found that the intermolecular bonds may prevent proper inflow/outflow of cations, and the pKa of the polypeptide is determined by the intermolecular bonds between the polypeptides controlled by the arrangement of hydrophobic side chains.

### [Example 4] Analysis of Physicochemical Properties and mRNA Transfer Efficiency of Polyplexes

4-1. Analysis of Free mRNA Bands in Accordance with the N/P Ratio by Polyplexes

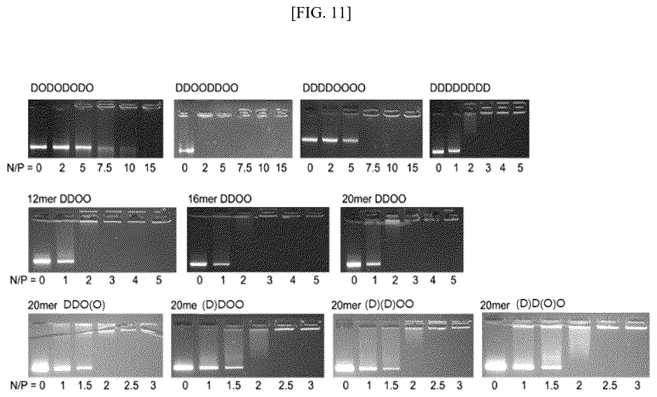

Polyplex samples were prepared by mixing FLuc-mRNA with 1929 nucleotides at various N/P ratios in 10 mM HEPES buffer (pH 7.3), and their complexes were detected by agarose gel electrophoresis (FIG. 11). Of the four octameric polypeptides, the free mRNA bands (mRNAs not bound to polymers) disappeared at N/P≥2 in DDDDDDDDDDD, which is believed to be due to the fact that the DET moiety on the side chain has a degree of amine protonation of about 50%, and the N/P of 2 corresponds to a charge neutralization point between the protonated amines on the DET moiety and the phosphate of FLUC-mRNA. In addition, DDOODDOO showed a similar association in mRNA with homopolypeptides whose free mRNA bands disappeared at N/P≥2. This is well consistent with the degree of protonation of DDOODDOO shown in FIG. 9A, indicating that the two repeating units of DET are sufficiently related to their counterparts. On the other hand, free mRNA bands disappeared at N/P≥15 and N/P≥7.5, respectively, in DODODODO and DDDDOOOO, which is determined to be due to the low amount of accessible protonated amino groups in the polypeptide. DODODO with one repeating unit of DET had the lowest binding affinity for mRNA, indicating the importance of alternative sequence arrangements for mRNA binding. In the case of long DDOO, the free mRNA band disappeared at N/P≥2, identifying that the degree of protonation of the polypeptide and its association with the mRNA were not affected by the length of the polypeptide.

4-2. Analysis of Hydrodynamic Diameter, Polydispersity Index, and Zeta Potential in Accordance with the N/P Ratio by Polyplexes

The hydrodynamic diameter (hereinafter referred to as DH), polydispersity index (hereinafter referred to as PDI), and zeta potential of the FLUC-mRNA-loaded polyplex sample prepared at various N/P ratios were measured with Zetasizer (Table 1). Delayed mRNA association of DODODODO and DDDDOOOO was also identified by zeta potential. As a result, at N/P=5, the zeta potential was −2.6±2.2 mV for DODODODO and −16±4.9 mV for DDDDOOOO, but +16±0 mV for DDOODDOO. In addition, the longer the DDOO polypeptide, the smaller the size of the polyplex. The hydrodynamic diameter of the 20 mer DDOO polyplex was found to be ~200 nm. The zeta potential of all polyplexes having 12 mer, 16 mer, and 20 mer DDOO polypeptides was found to be between +30 to +40 mV.

TABLE 1

| | N/P | | | Zeta-potential |
|---|---|---|---|---|
| Polypeptides | ratio | D_{H} (nm) | PDI | (mV) |
| DODODODO | 5 | 450 ± 360 | 0.55 ± 0.17 | −2.6 ± 2.2 |
| | 10 | 450 ± 200 | 0.57 ± 0.08 | −3.0 ± 9.0 |
| | 20 | 3200 ± 660 | 0.55 ± 0.17 | −1.1 ± 3.3 |
| | 30 | 4330 ± 430 | 0.35 ± 0.13 | 0.1 ± 6.6 |
| DDOODDOO | 5 | 5720 ± 2010 | 0.45 ± 0.05 | 16 ± 0 |
| | 10 | 950 ± 570 | 0.88 ± 0.09 | 38 ± 4.7 |
| | 20 | 400 ± 30 | 0.86 ± 0.04 | 38 ± 4.4 |
| | 30 | 460 ± 200 | 0.51 ± 0.07 | 39 ± 4.1 |
| DDDDOOOO | 5 | 370 ± 110 | 0.54 ± 0.11 | −16 ± 4.9 |
| | 10 | 400 ± 40 | 0.59 ± 0.07 | 9.0 ± 6.3 |
| | 20 | 2660 ± 950 | 0.60 ± 0.15 | 27 ± 3.9 |
| | 30 | 4280 ± 680 | 0.41 ± 0.06 | 38 ± 3.8 |
| 12 mer DDOO | 5 | 430 ± 210 | 0.46 ± 0.15 | 31 ± 4.3 |
| 16 mer DDOO | 5 | 130 ± 7 | 0.34 ± 0.06 | 36 ± 3.8 |
| 20 mer DDOO | 5 | 200 ± 20 | 0.36 ± 0.12 | 33 ± 4.3 |
| 20 mer DDO(0) | 5 | 6900 ± 1800 | 0.40 ± 0.13 | 26 ± 3.4 |
| 20 mer (D)DOO | 5 | 190 ± 20 | 0.55 ± 0.08 | 31 ± 4.0 |
| 20 mer (D)(D)OO | 5 | 200 ± 30 | 0.64 ± 0.15 | 31 ± 4.9 |
| 20 mer (D)D(0)O | 5 | 170 ± 25 | 0.43 ± 0.06 | 33 ± 5.1 |

*Table 1 above is expressed as mean ± SD (n = 5).
*In the experimental group, D or O in parentheses has D-form, and D or O not in parentheses has L-form. Parentheses are used for the sole purpose of indicating D-form.

4-3. Analysis of mRNA Transfer Efficiency (mRNA Translation Efficacy)

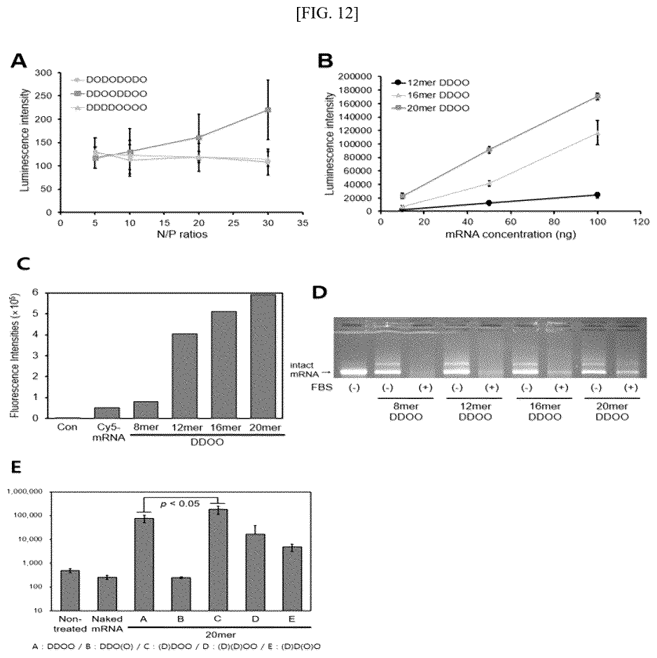

The IVT mRNA transfer efficiency of polyplexes was determined using A549 cells. Octameric polypeptides were mixed with FLuc-mRNA at various N/P ratios to produce polyplex samples. Polyplex samples were transfected into cells with 500 ng mRNA/well. Cells were cultured for 24 hours, and FLuc expression levels were measured with a luminescence plate reader (FIG. 12A). As a result, at N/P=30, DDOODDOO polyplexes had significantly higher luciferase expression levels than that of other polypeptide polyplexes, revealing that DDOODDOO with appropriate pKa for endosomal escape and hydrophobic moieties for high polyplex stability is essential for high mRNA translation efficacy.

In addition, experiments were performed on long DDOO polypeptides, in which 12 mer, 16 mer, and 20 mer DDOO polypeptides were mixed with FLuc-mRNA at N/P=5 to produce polyplex samples, and polyplex samples were transfected into A549 cells at various mRNA concentrations (FIG. 12B). As a result, the luminescence intensity of DDOO polyplex with longer length found to be 103~104 times higher than that of octameric DDOO polyplexes at N/P=30 (500 ng mRNA/well). Thereby, it was determined that polypeptide length affects the efficacy of mRNA transfection.

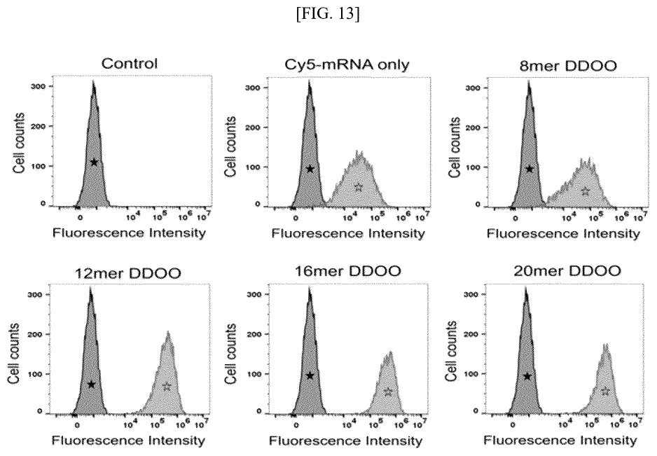

In addition, to explain the underlying mechanism of polypeptide length-dependent IVT mRNA expression, the cellular uptake of IVT mRNA was analyzed by flow cytometry using Cy5-labeled FLuc-mRNA (Cy5-mRNA). Prior to flow cytometry, A549 cells were cultured with naked Cy5-mRNA or Cy5-mRNA-loaded polyplex for 4 hours (FIG. 12C and FIG. 13). As a result, polyplexes using 12, 16 and 20 mer DDOO showed 5~7 times higher cellular uptake than polyplexes using DDOODDOO, identifying that polypeptide length affects cellular uptake.

In addition, stability of polyplexes (or IVT mRNA payload) was analyzed after culture with 10% FBS for 1 hour at 37° C. (FIG. 12D). The polyplex was then dissociated, and the intact IVT mRNA was visualized via agarose gel electrophoresis. As a result, higher amounts of intact IVT mRNA appeared in the longer DDOO polypeptide. Thereby, it was determined that the efficient cellular uptake of polyplexes is mainly due to its high tolerability in the FBS solution.

In addition, to determine the effect of isomers (L-form, D-form) on mRNA delivery efficiency, A549 cells were transfected with various polyplexes in which FLuc-mRNAs are included (FIG. 12E). As a result, 20 mer (D)DOO showed a luminescence intensity 2.4 times higher than that of 20 mer L-form DDOO, and from the above results, it was found that the isomer of the polypeptide affects the efficacy of mRNA delivery. In addition, 20 mer DDO(O) and (D)D(O)O showed a luminescence intensity 101~102 times lower than that of 20 mer (D)DOO, finding that insertion of D-Asp(OCT) significantly reduced mRNA translation efficiency.

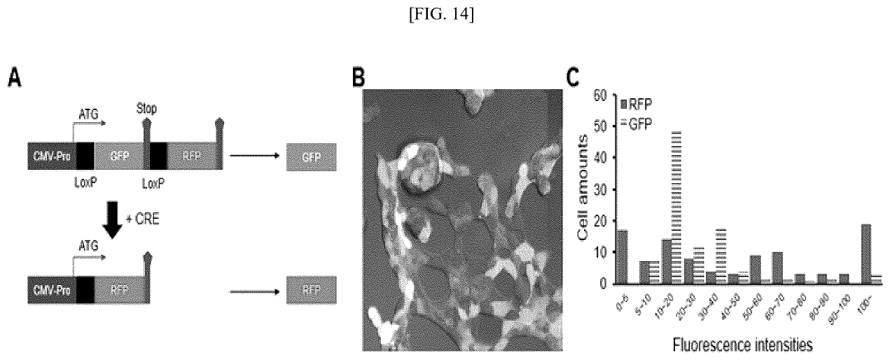

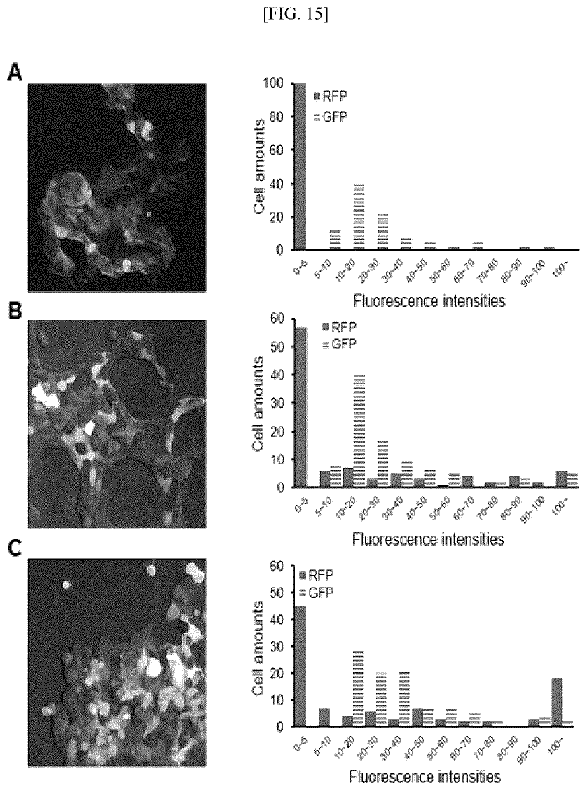

In addition, in vitro gene editing efficacy was analyzed using HEK293-loxP-GFP-RFP (Neo) cells exhibiting red fluorescence after LoxPs were cleaved via Cre recombinase (FIG. 14A). 20 mer DDOO and (D)DOO were mixed with Cre-mRNA to form polyplex samples at N/P=5. Lipofectamine 3000 was used as a positive control group. Samples were transfected into cells for 48 hours, and the cells were observed using CLSM. Of 100 randomly selected cells, the amount of GFP and/or RFP positive cells and the fluorescence intensity thereof were quantified. As a result, non-treated cells expressed a strong green fluorescence signal, but the downstream RFP did not (FIG. 15A). 20 mer DDOO showed higher gene editing efficacy than the 20 mer (D)DOO and positive control group. Specifically, the cells treated with 20 mer DDOO polyplexes exhibited 82 RFP-positive cells with reduced GPF fluorescence intensity (FIG. 14B and FIG. 14C). Cells treated with 20 mer (D)DOO polyplexes or positive control group showed 43 and 55 RFP-positive cells, respectively (FIG. 15B and FIG. 15C). From the above results, it was found that 20 mer DDOO exhibited a higher delivery efficacy for Cre-mRNA than commercially available transfection reagents, and the 20 mer DDOO exhibited a higher delivery efficacy for Cre-mRNA than the 20 mer (D)DOO, which is believed to be due to the difference in the mRNA length of FLuc- and Cre-mRNA (1929 and 1351 nt, respectively), that affects polyplex formation.

### [Example 5] Length-Dependent Polypeptide/RNA Complexation

Since the formation of supramolecular networks within mRNA polyplexes is important for improving transfection efficiency, the ssRNA complexation process was analyzed to understand the length-dependent efficacy of DDOO polyplexes. Given that single-stranded regions of mRNA have a highly flexible structure and are best suitable for polycation bundling, the first 20 nucleotides of Fluc mRNA were selected as single-stranded RNA (ssRNA) prototypes to study the complexation process. To assert the representativeness of the prototype, the physicochemical properties (DH, PDI, and zeta potential) of the polyplex loaded with ssRNA, similar to the polyplex loaded with FLuc-mRNA, were analyzed (Table 2).

TABLE 2

| | N/P | | | Zeta-potential |
|---|---|---|---|---|
| Polypeptides | ratio | D_{H} (nm) | PDI | (mV) |
| DODODODO | 5 | 260 ± 73 | 0.52 ± 0.15 | −33 ± 4 |
| | 10 | 570 ± 410 | 0.62 ± 0.22 | −19 ± 19 |
| | 20 | 1660 ± 247 | 0.47 ± 0.12 | −11 ± 7 |
| | 30 | 3180 ± 709 | 0.36 ± 0.14 | 0.3 ± 10 |
| DDOODDOO | 5 | 1430 ± 357 | 0.45 ± 0.13 | 5.8 ± 4 |
| | 10 | 700 ± 241 | 0.69 ± 0.10 | 14 ± 4 |
| | 20 | 468 ± 91 | 0.44 ± 0.05 | 27 ± 3 |
| | 30 | 449 ± 126 | 0.41 ± 0.10 | 28 ± 3 |
| DDDDOOOO | 5 | 8370 ± 3070 | 0.57 ± 0.14 | −0.2 ± 7 |
| | 10 | 5980 ± 1400 | 0.42 ± 0.09 | 24 ± 7 |
| | 20 | 1680 ± 626 | 0.92 ± 0.07 | 33 ± 16 |
| | 30 | 963 ± 253 | 0.87 ± 0.07 | 31 ± 11 |
| 12 mer DDOO | 5 | 220 ± 28 | 0.24 ± 0.02 | 20 ± 14 |
| 16 mer DDOO | 5 | 250 ± 31 | 0.30 ± 0.09 | 34 ± 11 |
| 20 mer DDOO | 5 | 260 ± 25 | 0.31 ± 0.04 | 33 ± 13 |

*Table 2 above is expressed as mean ± SD (n = 5).

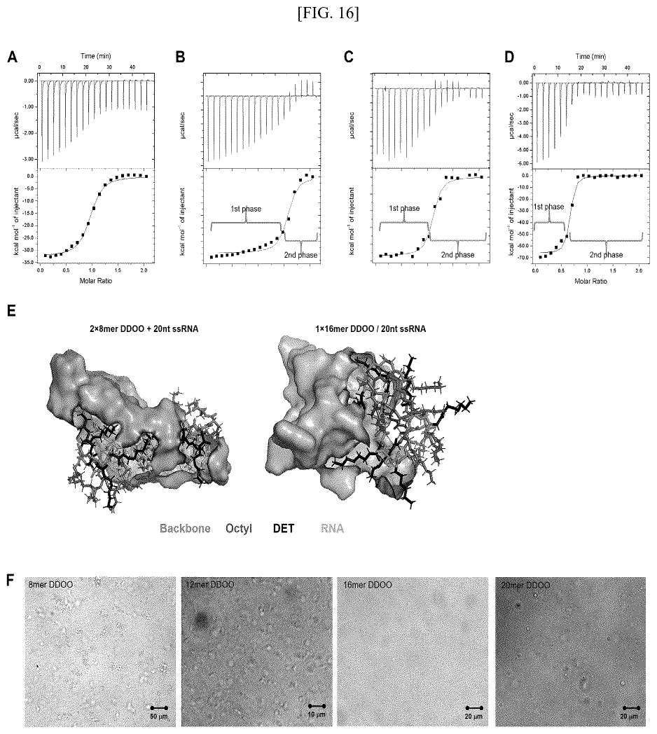

First, isothermal titration calorimetry (ITC) was used to analyze the complexation between DDOO polypeptides and ssRNAs. All DDOO polypeptides showed exothermic complexation with ssRNA (FIGS. 16A to 16D, Table 3). In addition, heat release (ΔH) increased when the length of the DDOO polypeptide increased from 8 mer to 12 mer, but there was no relatively significant change when the length increased from 16 mer to 20 mer. It was determined that, when sequence lengths exceed 8 mer, DDOO polypeptides may form more energy-stable complexes with ssRNAs. In addition to the energy stability, changes in the mechanism of ssRNA complexation were identified when the sequence length of the DDOO polypeptide exceeded 8 mer. Using a single-site binding model, a good fit was obtained for an experimental binding isotherm of an 8 mer DDOO/ssRNA pair with a binding stoichiometry (N value) of ~1 (FIG. 16A). It was determined that the complexation of 8 mer DDOO/ssRNA is a one-step process. On the other hand, experimental binding isotherms of longer DDOO/ssRNA pairs showed two-step energy transition (red in the first step and green in the second) (FIGS. 16B to 16D). The process of polyelectrolyte complexation in the above two steps has previously been confirmed by other studies, the first step corresponds to the spontaneous ion pairing of the oppositely charged group, while the second step corresponds to the formation of a supramolecular network from the ion pair. This difference in the mechanism was also reflected in the increase in the N value of 12 mer compared to 8 mer. In general, longer DDOO polypeptides have a higher number of cationic groups per molecule, resulting in lower N values when the concentration of an injectant (DDOO polypeptide) remains constant. This mechanistic difference was also observed in the decrease in the N value when the DDOO polypeptide was extended from 12 mer to 20 mer. However, when the length increased from 8 mer to 12 mer, the opposite pattern emerged, resulting in an increase in the N value, not a decrease. These higher-than-expected N values are believed to be attributed to the consumption of 12 mers in constructing a supramolecular network demonstrating mechanistic transition from the first step to the ssRNA complex in the second step when the DDOO polypeptide exceeds the length of 8 mers.

TABLE 3

| | | | ΔH | TΔS |
|---|---|---|---|---|
| Polyplexes | N | K (M⁻¹) | (kcal/mol) | (kcal/mol) |
| 8 mer DDOO/ | 0.93 ± 0.01 | 1.74 × 10⁶ ± | −32.4 ± 0.5 | −23.9 |
| 20 nt ssRNA | | 2.91 × 10⁵ | | |
| 12 mer DDOO/ | 1.53 ± 0.02 | 5.14 × 10⁶ ± | −59.4 ± 1.0 | −50.4 |
| 20 nt ssRNA | | 1.66 × 10⁶ | | |
| 16 mer DDOO/ | 1.07 ± 0.01 | 4.40 × 10⁶ ± | −46.7 ± 1.0 | −37.5 |
| 20 nt ssRNA | | 1.40 × 10⁶ | | |
| 20 mer DDOO/ | 0.63 ± 0.01 | 7.31 × 10⁶ ± | −66.1 ± 1.4 | −56.6 |
| 20 nt ssRNA | | 2.55 × 10⁶ | | |

Subsequently, AAMD was performed to explain the molecular basis for the length-dependent transition of the ssRNA complexation mechanism observed in ITC measurements. To support the experimental conditions, the same ssRNA sequence was used for the MD simulation. As a result of sampling MD simulations (100 ns) of 2×8 mer/ssRNA and 1×16 mer/ssRNA to compare the ssRNA complexation process between cationic polymers with the same sequence form ((DDOO) n) and residue numbers (8×OCT and 8×DET), it was found that they exist in two different molecular forms (2×8 mer versus 1×16 mer) (FIG. 16E). The conformation of the DDOO polypeptide was found to be largely unaffected after complexation with ssRNA, which is probably due to the stabilization of intramolecular hydrogen bonds imposed by multiple β-turns, such as the structure of the polypeptide (FIG. 9E). As a result of the undisturbed form of the polypeptide, it was observed that the cationic moiety, DET, was displayed in a defined pattern for ssRNA complexation (FIG. 16E). From these AAMD results, it may be hypothesized that 8 mer DDOOs, which have a dense molecular size due to their secondary structure, are more likely to induce ssRNA complexion (FIG. 16E). On the other hand, longer DDOO polypeptides with more extended molecular sizes may potentially act as cross-linkers for ssRNAs, aiding in the formation of supramolecular networks within the polyplex. Overall, AAMD showed that the form of DDOO polypeptides was stable when binding to ssRNA, demonstrating the molecular basis of the length-dependent mode of ssRNA complexation. Finally, analysis was conducted on how changes in the complexation mechanism between DDOO polypeptides and RNA affect the macroscopic properties of the complexes. Bright-field microscopy was used to image the DDOO/RNA complex at NP=5, that was used for in vitro mRNA transfection analysis.

As a result, micron-sized aggregates were observed only in the 8 mer/ssRNA complex, and no such aggregates were observed in the ssRNA complexes formed in the longer length of the DDOO polypeptide. These results indicate that when DDOO polypeptides form complexes with ssRNAs through a single ion pairing step, they tend to induce the formation of micron-sized aggregates. The result may be explained in a way that the micron-sized aggregates are formed as these charge-neutralized complexes undergo hydrophobic collapse in the absence of a supramolecular network (FIG. 16F). On the other hand, longer DDOO/ssRNA complexes with supramolecular networks may form a more condensed structure (FIG. 16F).

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the attached claims and their equivalents.

The invention claimed is:

1. A polypeptide polymer represented by the following Chemical Formula 1: 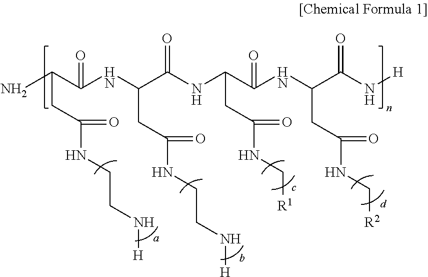
wherein, in the Chemical Formula 1,
n is one of 1 to 20,
a and b may be the same or different and are each independently one of 1 to 5,
c and d may be the same or different and are each independently one of 1 to 20, and
R¹ and R² may be the same or different and are each independently hydrogen or (C4-C12)cycloalkyl.

2. The polypeptide polymer of claim 1, wherein, in the Chemical Formula 1, n is one of 1 to 5, a and b may be the same or different and are each independently one of 1 to 3, c and d may be the same or different and are each independently one of 5 to 10, and R¹ and R² may be the same or different and are each independently hydrogen or (C4-C8)cycloalkyl.

3. The polypeptide polymer of claim 1, wherein, in the Chemical Formula 1, n is one of 1 to 5, a and b are each independently one of 1 to 3, c and d are each independently one of 5 to 10, and R¹ and R² are each independently hydrogen or (C4-C8)cycloalkyl.

4. The polypeptide polymer of claim 3, wherein the polymer is in a form in which diethylenetriamine (DET) or octylamine (OCT) is bonded to aspartic acid.

5. The polypeptide polymer of claim 4, wherein the form in which diethylenetriamine (DET) or octylamine (OCT) is bonded to aspartic acid has an L-form or D-form.

6. The polypeptide polymer of claim 1, wherein the polypeptide polymer increases RNA transcription efficiency.

7. The polypeptide polymer of claim 6, wherein the RNA is one or more selected from the group consisting of mRNA, siRNA, antisense oligonucleotide (ASO), and guide RNA (gRNA).

8. The polypeptide polymer of claim 7, wherein the mRNA is an in vitro transcribed (IVT) mRNA.

9. A RNA delivering system, comprising the polypeptide polymer of claim 1.

10. A method of promoting RNA transcription, in a subject in need thereof, comprising:
administering the polypeptide polymer of claim 1 to the subject.

11. A method for preparing the polypeptide polymer of claim 1, the method comprising:
preparing a conjugate in which diethylenetriamine (DET) is bonded to aspartic acid (first step);
preparing a conjugate in which octylamine (OCT) is bonded to aspartic acid (second step); and
binding the conjugate of the first step and the conjugate of the second step (third step).
